(19) United States
(12) Patent Application Publication
(10) Pub. No.: US 2021/0307810 A1
(43) Pub. Date: Oct. 7, 2021

(54) SYSTEM AND METHOD FOR SEALING TISSUE

(71) Applicant: Covidien LP, Mansfield, MA (US)

(72) Inventor: Richard L. Croft, Mead, CO (US)

(21) Appl. No.: 17/189,322

(22) Filed: Mar. 2, 2021

Related U.S. Application Data
(60) Provisional application No. 63/004,736, filed on Apr. 3, 2020.

(51) Int. Cl. A61B 18/14

(52) U.S. Cl. CPC A61B 18/1442; A61B 2018/0063

(57) ABSTRACT

An electrosurgical system includes an electrosurgical generator and an electrosurgical instrument coupleable to the electrosurgical generator. The electrosurgical generator includes a controller, a power supply coupled to the controller, and a power converter coupled to the power supply. The controller is configured to cause the power converter to generate a pulsed electric field configured to electroporate tissue and RF current configured to seal tissue. The electrosurgical instrument includes a pair of opposing jaw members configured to grasp tissue. Each of the pair of opposing jaw members includes an electrically conductive tissue-contacting surface configured to electroporate tissue disposed in proximity to the electrically conductive tissue-contacting surfaces via the pulsed electric field and deliver the RF current to the electroporated tissue to seal the electroporated tissue.

### CROSS REFERENCE TO RELATED APPLICATION

This application claims the benefit of the filing date of provisional U.S. application Ser. No. 63/004,736, filed on Apr. 3, 2020.

### INTRODUCTION

The present disclosure relates to systems and methods for sealing tissue. In particular, the present disclosure relates to an electrosurgical system including a generator coupleable with an electrosurgical instrument for electroporating tissue by applying a pulsed, high voltage electric field to an end effector of the electrosurgical instrument and subsequently delivering radio frequency (“RF”) current to the electroporated tissue via the end effector to seal the electroporated tissue.

### BACKGROUND

Electrosurgery involves application of RF electrical current to a surgical site to cut, ablate, desiccate, or coagulate tissue. In monopolar electrosurgery, a source or active electrode delivers radio frequency alternating current from the electrosurgical generator to the targeted tissue. A patient return electrode is placed remotely from the active electrode to conduct the current back to the generator.

Bipolar electrosurgery generally involves the use of forceps. A forceps is a pliers-like instrument which relies on mechanical action between its jaws to grasp, clamp and constrict vessels or tissue. So-called “open forceps” are commonly used in open surgical procedures whereas “endoscopic forceps” or “laparoscopic forceps” are, as the name implies, used for less invasive endoscopic surgical procedures. Electrosurgical forceps (open or endoscopic) utilize mechanical clamping action and electrical energy to effect hemostasis on the clamped tissue. The forceps include electrodes that apply the electrosurgical energy to the clamped tissue. By controlling the intensity, frequency, and duration of the electrosurgical energy applied to tissue through the electrodes to the tissue, the surgeon can coagulate, cauterize, and/or seal tissue. Tissue or vessel sealing is a process of liquefying the collagen, elastin, and ground substances in the tissue so that they reform into a fused mass with significantly-reduced demarcation between the opposing tissue structures.

Another electrosurgical technique is electroporation in which electric field pulses are applied across the tissue cells to generate a destabilizing electric field across cells' outer membrane and cause the formation of nanoscale defects in the lipid bilayer of the cells. The nanoscale defects (e.g., pores) created in the cell membrane allows for water within the cell to escape, resulting in tissue dehydration. Electroporating tissue prior to sealing the tissue may have positive effects on a subsequent tissue sealing procedure performed on the porated tissue due to the reduced cellular water content of the electroporated tissue. Reduced cellular water content in tissue to be sealed may result in a reduction in the level of treatment parameters (e.g., power output, clamping force, procedure time) required for achieving an effective tissue seal, thereby helping to minimize adverse thermal effects (e.g., thermal spread) associated with conventional tissue sealing procedures and allow for sealing of larger vessels, which may typically require higher power output and longer sealing times relative to smaller vessels.

### SUMMARY

According to one embodiment of the present disclosure, an electrosurgical system includes an electrosurgical generator and an electrosurgical instrument coupleable to the electrosurgical generator. The electrosurgical generator includes a controller, a power supply coupled to the controller, and a power converter coupled to the power supply. The controller is configured to cause the power converter to generate a pulsed electric field configured to electroporate tissue and RF current configured to seal tissue. The electrosurgical instrument includes a pair of opposing jaw members configured to grasp tissue. Each jaw of the pair of opposing jaw members includes an electrically conductive tissue-contacting surface configured to electroporate tissue disposed in proximity to the electrically conductive tissue-contacting surfaces via the pulsed electric field and deliver the RF current to the electroporated tissue to seal the electroporated tissue.

According to one aspect of the above embodiment, the power converter includes an oscillator configured to generate an RF signal.

According to another aspect of the above embodiment, the power converter includes a modulator circuit configured to gate the RF signal generated by the oscillator to generate an RF pulse.

According to yet another aspect of the above embodiment, the power converter includes an amplifier configured to amplify the RF pulse generated by the modulator circuit and output the amplified RF pulse to the electrosurgical instrument for applying the pulsed electric field to the tissue.

According to yet another aspect of the above embodiment, the power converter is configured to adjust a voltage amplitude duration of the pulsed electric field to control a degree of electroporation of the tissue.

According to yet another aspect of the above embodiment, the power converter is configured to adjust a shape of the pulsed electric field to control a degree of electroporation of the tissue.

According to yet another aspect of the above embodiment, the power converter is configured to adjust a number of pulses of the pulsed electric field to control a degree of electroporation of the tissue.

According to yet another aspect of the above embodiment, the controller is configured to signal the power converter to generate the RF current subsequent to generating the pulsed electric field configured to electroporate the tissue.

According to another embodiment of the present disclosure, an electrosurgical generator includes a controller, a power supply coupled to the controller, and a power converter coupled to the power supply. The controller is configured to cause the power converter to output, to an electrosurgical instrument coupled to an output of the power converter, a pulsed electric field configured to electroporate tissue disposed in proximity to an end effector of the electrosurgical instrument and RF current configured to treat the electroporated tissue.

According to one aspect of the above embodiment, the power converter includes an oscillator configured to generate an RF signal.

According to another aspect of the above embodiment, the power converter includes a modulator circuit configured to gate the oscillating RF signal generated by the RF oscillator to generate an RF pulse.

According to yet another aspect of the above embodiment, the power converter includes an amplifier configured to amplify the RF pulse generated by the modulator circuit and output the amplified RF pulse to an electrosurgical instrument coupled to the output of the power converter.

According to yet another aspect of the above embodiment, the pulsed electric field is an oscillating pulsed RF electric field including a DC offset.

According to yet another aspect of the above embodiment, the power converter is configured to adjust a voltage amplitude duration of the pulsed electric field to control a degree of electroporation of the tissue.

According to yet another aspect of the above embodiment, the power converter is configured to adjust a shape of the pulsed electric field to control a degree of electroporation of the tissue.

According to yet another aspect of the above embodiment, the power converter is configured to adjust a number of pulses of the pulsed electric field to control a degree of electroporation of the tissue.

According to yet another embodiment of the present disclosure, a method for sealing tissue includes positioning at least one electrode of an electrosurgical instrument in proximity to tissue, applying a pulsed electric field to the at least one electrode of the electrosurgical instrument to electroporate the tissue, grasping the electroporated tissue between a pair of jaw members of the electrosurgical instrument, and delivering RF current to the grasped electroporated tissue via the at least one electrode of the electrosurgical instrument to seal the electroporated tissue.

According to one aspect of the above embodiment, the method also includes adjusting a voltage amplitude duration of the applied pulsed electric field to control a degree of electroporation of the tissue.

According to another aspect of the above embodiment, the method also includes adjusting a pulse shape of the applied pulsed electric field to control a degree of electroporation of the tissue.

According to yet another aspect of the above embodiment, the method also includes adjusting a number of pulses of the applied pulsed electric field to control a degree of electroporation of the tissue.

### BRIEF DESCRIPTION OF THE DRAWINGS

The present disclosure may be understood by reference to the accompanying drawings, when considered in conjunction with the subsequent, detailed description, in which:

FIG. 1 is a perspective view of a shaft-based electrosurgical forceps provided in accordance with the present disclosure connected to an electrosurgical generator;

FIG. 2A is a perspective view of a distal end portion of the forceps of FIG. 1, wherein jaw members of an end effector assembly of the forceps are disposed in a spaced-apart position;

FIG. 2B is a perspective view of the distal end portion of the forceps of FIG. 1, wherein the jaw members are disposed in an approximated position;

FIG. 3 is a perspective view of a hemostat-style electrosurgical forceps provided in accordance with the present disclosure;

FIG. 4 is a schematic illustration of a robotic surgical instrument provided in accordance with the present disclosure;

FIG. 5 is a block diagram of the electrosurgical generator of FIG. 1;

FIG. 6 is a block diagram of a power converter of the electrosurgical generator of FIG. 1; and

FIG. 7 is a flow chart of a method for sealing tissue according to an embodiment of the present disclosure.

### DETAILED DESCRIPTION

Particular embodiments of the present disclosure will be described below with reference to the accompanying drawings. In the following description, well-known functions or constructions are not described in detail to avoid obscuring the present disclosure in unnecessary detail. Those skilled in the art will understand that the illustrative embodiments of the present disclosure may be adapted for use with any electrosurgical instrument. It should also be appreciated that different electrical and mechanical connections and other considerations from those described in the present disclosure may apply to each particular type of instrument.

The present disclosure provides an electrosurgical system including a generator that can generate pulsed, high voltage electric fields to electroporate target tissue via any suitable electrosurgical instrument coupleable to the generator and configured to apply the pulsed high voltage electric fields to the target tissue. In an embodiment of the present disclosure, a suitable electrosurgical instrument (e.g., an electrosurgical forceps) is coupleable to the generator and includes a pair of opposing jaw members each having an electrically conductive tissue-contacting surface (e.g., an electrode) to which the generator may apply a pulsed, high voltage electric field for electroporating tissue in proximity to at least one of the opposing tissue-contacting surfaces of the electrosurgical instrument and/or tissue grasped between the opposing tissue-contacting surfaces of the electrosurgical instrument. In one aspect of the embodiments of the present disclosure, one or both jaw members of the pair of opposing jaw members may include an additional electrode or electrodes to which the generator applies the electric field for electroporating tissue in proximity to the additional electrode or electrodes. The generator may apply the electric field to the additional electrode(s) in lieu of the tissue-contacting surfaces, to the additional electrode(s) and one or both of the tissue-contacting surfaces, to one of the tissue-contacting surfaces only, or any suitable combination of the additional electrode(s) and tissue-contacting surfaces. In aspects of the present disclosure, the number of electrodes and/or tissue-contacting surfaces used to electroporate tissue may be varied to adjust the strength of the electric field and, as a result, the degree of electroporation of tissue. For example, the strength of the electric field may be increased by increasing the number of electrodes and/or tissue-contacting surfaces to which the electric field is applied by the generator.

In one aspect of the embodiments of the present disclosure, the electric field may be an oscillating pulsed RF electric field. In another aspect, the electric field may be an oscillating pulsed RF electric field with a DC offset. The generator can also generate and deliver RF current via the opposing tissue-contacting surfaces to seal tissue grasped therebetween. In an embodiment of the present disclosure, following electroporation of the tissue via application of a pulsed, high voltage electric field, the electrosurgical instrument is used to provide RF current to the electroporated tissue grasped between the opposing tissue-contacting surfaces to seal the electroporated tissue. In embodiments of the present disclosure, the generator may generate pulsed, high voltage electric fields and RF current either separately, simultaneously, or intermittently.

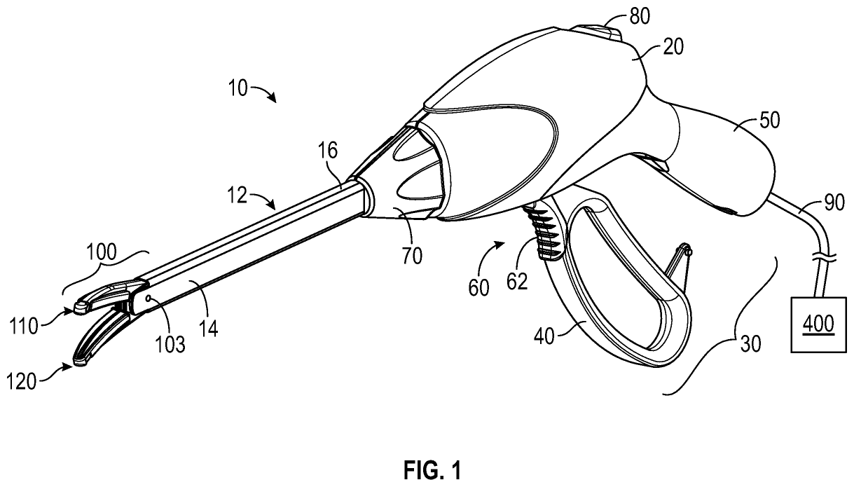

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

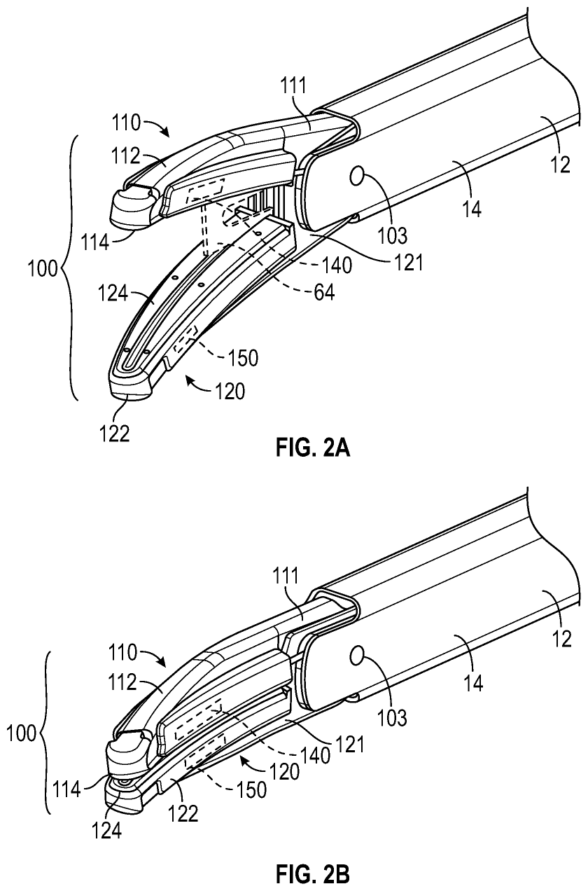

Forceps 10 includes a housing 20, a handle assembly 30, a trigger assembly 60, a rotating assembly 70, an activation switch 80, and an end effector assembly 100. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable 90 that connects forceps 10 to an electrosurgical generator 400. Cable 90 includes a wire (or wires) (not shown) extending therethrough that has sufficient length to extend through shaft 12 in order to provide energy to one or both tissue-contacting surfaces 114, 124 of jaw members 110, 120, respectively, of end effector assembly 100 (see FIGS. 2A and 2B). Activation switch 80 is coupled to tissue-contacting surfaces 114, 124 (FIGS. 2A and 2B) and electrosurgical generator 400 for enabling the selective activation of the supply of energy to jaw members 110, 120 for sealing tissue. Forceps 10 may be adapted for use with an energy modality that heats tissue including, but not limited to, RF current, light energy, and ultrasonic energy. Forceps 10 is also configured to generate an oscillating RF electric field to electroporate target tissue.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position (FIG. 2A) and an approximated position (FIG. 2B) to grasp tissue between jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120 (FIG. 2B).

Trigger assembly 60 includes a trigger 62 coupled to housing 20 and movable relative thereto between an un-actuated position and an actuated position. Trigger 62 is operably coupled to a knife 64 (FIG. 2A), so as to actuate knife 64 (FIG. 2A) to cut tissue grasped between jaw members 110, 120 of end effector assembly 100 upon actuation of trigger 62. As an alternative to knife 64, other suitable mechanical, electrical, or electromechanical cutting mechanisms (stationary or movable) are also contemplated.

With additional reference to FIGS. 2A and 2B, end effector assembly 100, as noted above, includes first and second jaw members 110, 120. Each jaw member 110, 120 includes a proximal flange portion 111, 121, an outer insulative jaw housing 112, 122 disposed about the distal portion (not explicitly shown) of each jaw member 110, 120, and a tissue-contacting surface 114, 124 (e.g., electrode), respectively. Proximal flange portions 111, 121 are pivotably coupled to one another about pivot 103 for moving jaw members 110, 120 between the spaced-apart and approximated positions, although other suitable mechanisms for pivoting jaw members 110, 120 relative to one another are also contemplated. The distal portions (not explicitly shown) of the jaw members 110, 120 are configured to support jaw housings 112, 122, and tissue-contacting surfaces 114, 124, respectively, thereon.

Outer insulative jaw housings 112, 122 of jaw members 110, 120 support and retain tissue-contacting surfaces 114, 124 on respective jaw members 110, 120 in opposed relation relative to one another. Tissue-contacting surfaces 114, 124 are at least partially formed from an electrically conductive material, e.g., for conducting electrical energy therebetween for electroporating and/or sealing tissue, although tissue-contacting surfaces 114, 124 may additionally or alternatively be configured to conduct any suitable energy, e.g., thermal, microwave, light, ultrasonic, sonication, etc., through tissue grasped therebetween for energy-based tissue sealing. As mentioned above, tissue-contacting surfaces 114, 124 are coupled to activation switch 80 and electrosurgical generator 400, e.g., via the wires (not shown) extending from cable 90 through forceps 10, such that energy may be selectively supplied to tissue-contacting surface 114 and/or tissue-contacting surface 124 and conducted therebetween and through tissue disposed between jaw members 110, 120 to seal tissue. In some embodiments of the present disclosure, one or both of jaw members 100, 120 may include an additional electrode or electrodes (e.g., electrodes 140, 150 shown in FIGS. 2A and 2B) electrically coupled to generator 400 and configured to electroporate tissue. Electrodes 140, 150 may be configured to conduct any suitable energy type as indicated above with respect to tissue-contacting surfaces 114, 124. It should be understood that electrodes 140, 150 are illustrative only in that each of jaw members 110, 120 may include a plurality of electrodes disposed in any suitable location along jaw members 110, 120.

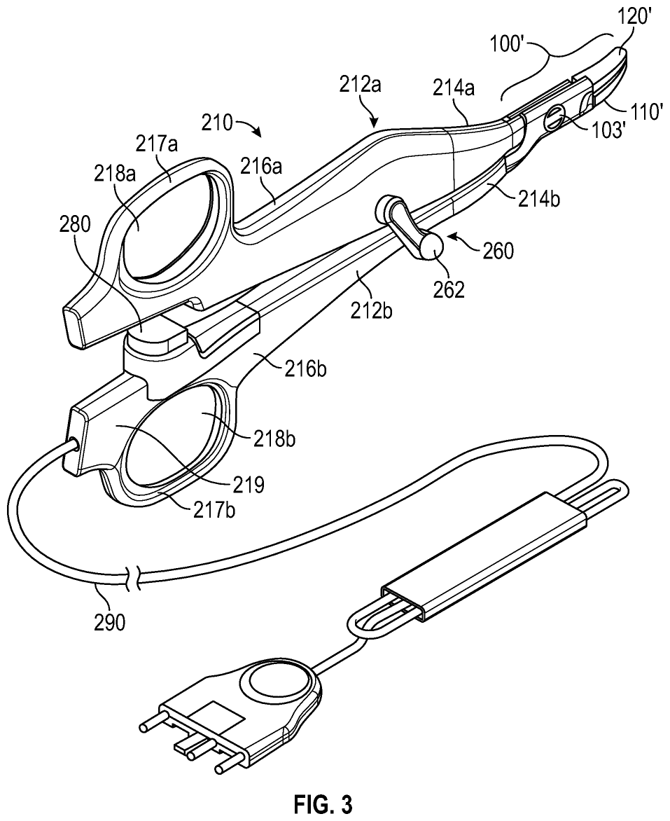

Referring to FIG. 3, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIGS. 2A and 2B). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to electrosurgical generator 400 (FIG. 1). Proximal shaft connector 219 secures a cable 290 to forceps 210 such that the user may selectively supply energy to jaw members 110′, 120′ for sealing tissue. More specifically, an activation switch 280 is provided for supplying energy to jaw members 110′, 120′ to seal tissue upon sufficient approximation of shaft members 212a, 212b, e.g., upon activation of activation switch 280 via shaft member 212a.

Forceps 210 further includes a trigger assembly 260 including a trigger 262 coupled to one of the shaft members, e.g., shaft member 212a, and movable relative thereto between an un-actuated position and an actuated position. Trigger 262 is operably coupled to a knife (not shown; similar to knife 64 (FIG. 2A) of forceps 10 (FIG. 1)) so as to actuate the knife to cut tissue grasped between jaw members 110,′ 120′ of end effector assembly 100′ upon movement of trigger 262 to the actuated position. Similarly as noted above with respect to forceps 10 (FIG. 1), other suitable cutting mechanisms are also contemplated.

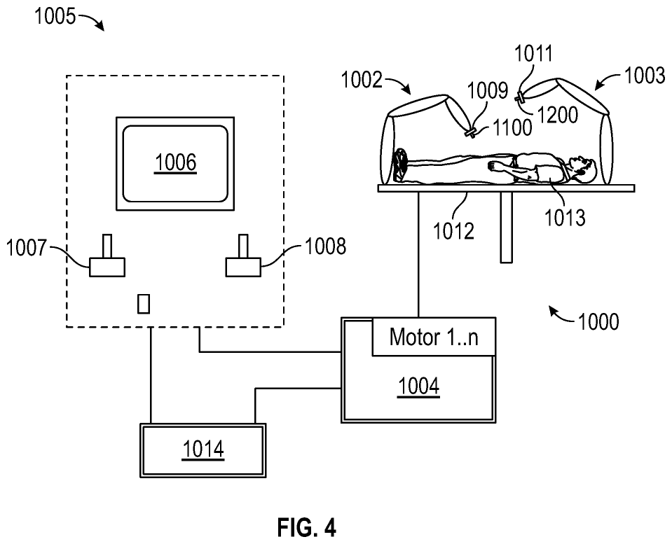

Referring to FIG. 4, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical instrument 1000 may be configured for use on a patient 1013 lying on a patient table 1012 to be treated in a minimally invasive manner. Robotic surgical instrument 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assembly 100 (FIGS. 2A and 2B), although other suitable end effector assemblies for coupling to attaching device 1009 are also contemplated. End effector assembly 1100 is connected to electrosurgical generator 400 (FIG. 1), which may be integrated into or separate from robotic surgical instrument 1000. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

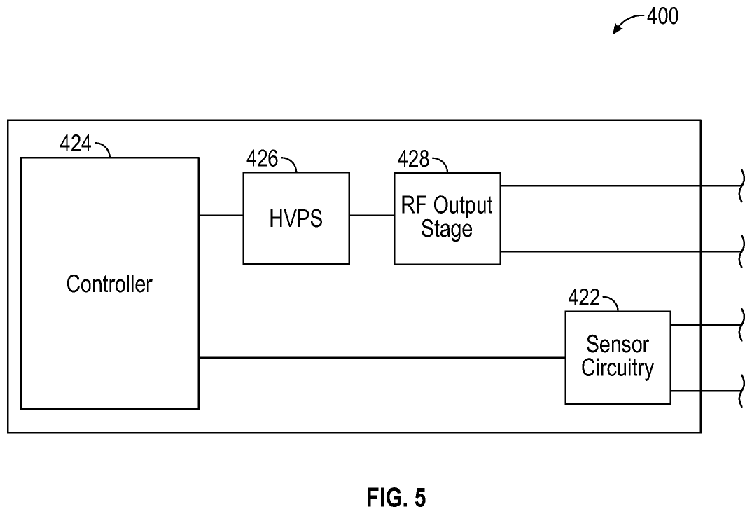

Referring to FIG. 5, electrosurgical generator 400 is shown as a schematic block diagram. Generator 400 may be utilized as a stand-alone generator (as shown in FIG. 1), may be incorporated into a surgical instrument 10, 210, 1000 (FIGS. 1, 3, and 4, respectively), or may be provided in any other suitable manner. Generator 400 generally includes a controller 424, a high voltage DC power supply (“HVPS”) 426, and a power converter 428. The HVPS 426 may be a high voltage, DC power supply connected to an AC source (e.g., line voltage) and provides high voltage, DC power to the power converter 428, which then converts high voltage, DC power into RF energy and delivers the energy to an electrosurgical instrument (e.g., forceps 10 or forceps 210) coupled to the generator 400.

HVPS 426, under the direction of controller 424, provides high voltage DC power to power converter 428 which converts the high voltage DC power into RF current for delivery to tissue-contacting surfaces 114, 124 of jaw members 110, 120, respectively, of end effector assembly 100 (see FIGS. 2A and 2B). In particular, power converter 428 generates sinusoidal waveforms of high frequency RF current. Power converter 428 may be configured to generate waveforms having various duty cycles, peak voltages, crest factors, and other parameters. Other suitable configurations are also contemplated such as for example, pulsed energy output, other waveforms, etc.

The controller 424 includes a processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to perform the calculations and/or set of instructions described herein.

The controller 424 includes an output port (not shown) that is operably connected to the HVPS 426 and/or power converter 228 allowing the processor to control the output of the generator 400 according to either open and/or closed control loop schemes. A closed loop control scheme is a feedback control loop, in which a plurality of sensors (not shown) measure a variety of tissue and energy properties (e.g., tissue impedance, tissue temperature, output power, current and/or voltage, etc.), and provide feedback to the controller 424. The controller 424 then controls the HVPS 426 and/or power converter 428, which adjusts the DC power and/or RF power, respectively.

The generator 400 according to the present disclosure may also include sensor circuitry 422 having one or more sensors coupled to the HVPS 426, controller 424, and/or power converter 428. The sensor circuitry 422 may be configured to sense properties of DC current supplied to the power converter 428 and/or the output of the power converter 428. Various components of the generator 400, e.g., the power converter 428 and/or the sensor circuitry 422, may be disposed on a printed circuit board (PCB). The controller 424 also receives input signals from the input controls of the generator 400 and the forceps 10, 210. The controller 424 utilizes the input signals to adjust power outputted by the generator 400 and/or performs other control functions thereon. The controller 424 may also utilize the input signals to control the degree of tissue electroporation.

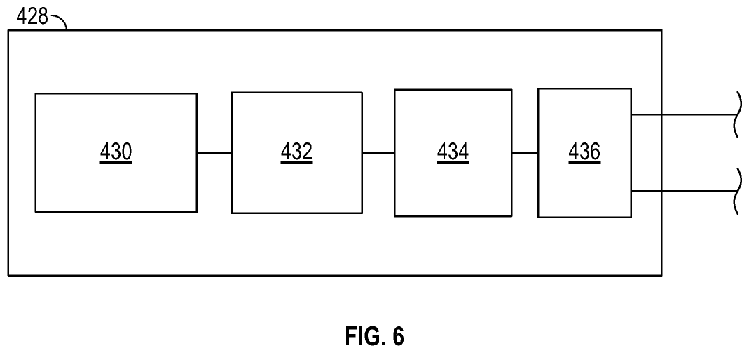

Referring to FIG. 6, the power converter 428 according to some embodiments of the present disclosure may include a power amplifier 436, a pulse controller 434, an oscillator 430 and a modulator circuit 432. The oscillator 430 is configured to generate a periodic, oscillating RF signal. The modulator circuit 432 serves to gate the RF signal output of the oscillator 430 to provide an RF pulse that may be controlled by the pulse controller 434 to control the degree of electroporation of tissue and that is amplified by the power amplifier 436 and output to the electrosurgical instrument (e.g., forceps 10 or forceps 210) coupled to the generator 400. The RF pulse output by the amplifier 436 is used to generate and apply a pulsed, high voltage electric field to the tissue-contacting surfaces 114, 124 or electrodes 140, 150 of jaw members 110, 120, respectively, of end effector assembly 100 (see FIGS. 2A and 2B) to electroporate tissue in proximity to the jaw members 110, 120 and/or tissue grasped between the tissue-contacting surfaces 114, 124. The controller 424 is configured to operate the power converter 428 to generate the pulsed, high voltage electric field and RF current either separately, simultaneously, or intermittently. The voltage of the electric field may be varied by the controller 424 to achieve various cellular effects. In some embodiments, the voltage of the electric field may be provided at a level sufficient to permanently damage the cellular membrane (e.g., irreversible electroporation (“IRE”)) or otherwise form pores therein.

If the strength of the electric field exceeds what is known as reversible threshold and exposure to tissue is of sufficient duration, so-called reversible electroporation occurs. In this scenario, the membrane is permeabilized and remains in a state of higher permeability for a period of time, but is eventually able to spontaneously return to its original state through a process of membrane resealing by which the pores close and the cell restores its normal transmembrane potential. If the strength of the electric field is sufficiently high, IRE occurs, resulting in loss of cell homeostasis, effectively killing the cell. In aspects of this disclosure, the pulse controller 434 may control the degree of electroporation of tissue by adjusting the voltage amplitude duration of the signal output of the oscillator 430, the pulse shape of the RF pulse output by the modulator circuit 432, and/or the number of RF pulses output by the modulator circuit 432, resulting in varying degrees of cellular effect ranging from reversible electroporation to irreversible electroporation. For example, in some embodiments the pulse controller 434 may control the power converter 428 to generate a high frequency IRE waveform, commonly referred to as “HFIRE.”

The pores created in the cell membrane allows for water within the cell to escape, resulting in tissue dehydration. Thus, electroporating tissue prior to sealing the tissue may have positive effects on a subsequent tissue sealing procedure performed on the porated tissue due to the reduced cellular water content. In particular, by reducing the cellular water content in the tissue to be sealed prior to the tissue sealing procedure, various parameters required to achieve an effective tissue seal may be reduced such as, for example, the power output of the generator 400, the clamping force applied to the tissue by the tissue-contacting surfaces 114, 124, and the length of time of the tissue sealing procedure. This reduction in power output, clamping force, and/or tissue sealing procedure time may help to minimize adverse thermal effects (e.g., thermal spread) associated with conventional tissue sealing procedures and allow for sealing of larger vessels, which may typically require higher power output and longer sealing times relative to smaller vessels, and sealing in areas of relatively increased sensitivity such as the lungs. Additionally, a reduction in power output, clamping force, and/or tissue sealing procedure time may result in less damage to collagen in the tissue being sealed, which will improve burst pressure performance of the sealed tissue (e.g., the maximum pressure at which the seal will fail). Electroporating tissue after or during sealing of the tissue may also result in an increased elasticity of the sealed tissue, which allows for expansion and contraction of the sealed tissue when under pressure to aid in preserving the integrity of the tissue seal.

Following electroporation of tissue via at least one of the tissue-contacting surfaces 114, 124, the electrodes 140, 150, or any combination thereof, the tissue-contacting surfaces 114, 124 of jaw members 110, 120, respectively, grasp and seal the electroporated tissue or, in the case where the tissue was electroporated while grasped between the tissue-contacting surfaces 114, 124, maintain a grasp on the electroporated tissue and seal the electroporated tissue. In some embodiments, RF current may be delivered prior to and/or intermittently during electroporation of the tissue (e.g., to pre-heat the tissue).

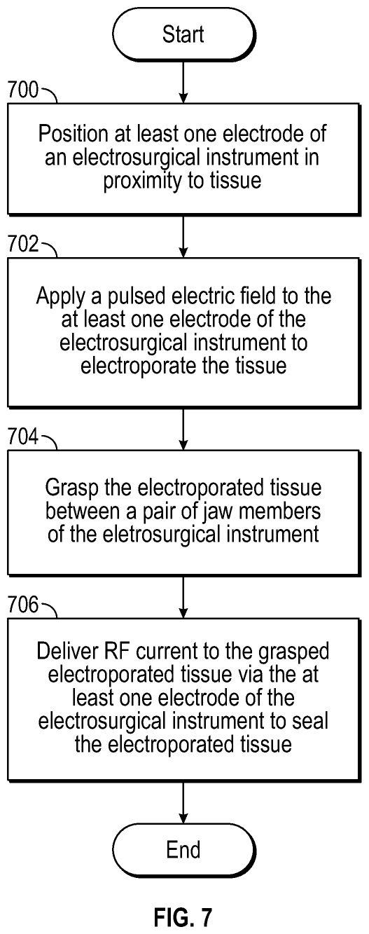

Referring now to FIG. 7, a method for sealing tissue begins at step 700, wherein an electrosurgical forceps (e.g., forceps 10 or forceps 210) is positioned in proximity to target tissue. This may be accomplished using various imaging modalities, such as X-ray, computer aided tomography, positron emission tomography, and the like. In step 702, a pulsed, high voltage electric field is applied to the electrosurgical instrument (e.g., forceps 10 or forceps 210) to electroporate the target tissue, thereby reducing the cellular water content of the target tissue. For example, the electric field may be applied to any combination of tissue-contacting surfaces 114, 124 and electrodes 140, 150 to effect electroporation of the target tissue. In step 704, RF current is applied to the electroporated target tissue via the electrosurgical instrument (e.g., forceps 10 or forceps 210) to treat the target tissue. For example, the electroporated tissue may be grasped between tissue-contacting surfaces 114, 124 and sealed via the application of RF current (e.g., bipolar, monopolar, etc.) through the grasped tissue.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical system, comprising:
an electrosurgical generator including:
a controller;
a power supply coupled to the controller; and
a power converter coupled to the power supply, the controller configured to cause the power converter to generate:
a pulsed electric field configured to electroporate tissue; and
RF current configured to seal tissue;
an electrosurgical instrument coupleable to the electrosurgical generator and including a pair of opposing jaw members configured to grasp tissue, each of the pair of opposing jaw members including an electrically conductive tissue-contacting surface configured to:
electroporate tissue disposed in proximity to the electrically conductive tissue-contacting surfaces via the pulsed electric field; and
deliver the RF current to the electroporated tissue to seal the electroporated tissue.

2. The electrosurgical system according to claim 1, wherein the power converter includes an oscillator configured to generate an RF signal.

3. The electrosurgical system according to claim 2, wherein the power converter includes a modulator circuit configured to gate the RF signal generated by the oscillator to generate an RF pulse.

4. The electrosurgical system according to claim 3, wherein the power converter includes an amplifier configured to amplify the RF pulse generated by the modulator circuit and output the amplified RF pulse to the electrosurgical instrument for applying the pulsed electric field to the tissue.

5. The electrosurgical system according to claim 1, wherein the power converter is configured to adjust a voltage amplitude duration of the pulsed electric field to control a degree of electroporation of the tissue.

6. The electrosurgical system according to claim 1, wherein the power converter is configured to adjust a shape of the pulsed electric field to control a degree of electroporation of the tissue.

7. The electrosurgical system according to claim 1, wherein the power converter is configured to adjust a number of pulses of the pulsed electric field to control a degree of electroporation of the tissue.

8. The electrosurgical generator according to claim 1, wherein the controller is configured to signal the power converter to generate the RF current subsequent to generating the pulsed electric field configured to electroporate the tissue.

9. An electrosurgical generator, comprising:
a controller;
a power supply coupled to the controller;
a power converter coupled to the power supply, the controller configured to cause the power converter to output to an electrosurgical instrument coupled to an output of the power converter:
a pulsed electric field configured to electroporate tissue disposed in proximity to an end effector of the electrosurgical instrument; and
RF current configured to treat the electroporated tissue.

10. The electrosurgical generator according to claim 9, wherein the power converter includes an oscillator configured to generate an RF signal.

11. The electrosurgical generator according to claim 10, wherein the power converter includes a modulator circuit configured to gate the oscillating RF signal generated by the RF oscillator to generate an RF pulse.

12. The electrosurgical generator according to claim 11, wherein the power converter includes an amplifier configured to amplify the RF pulse generated by the modulator circuit and output the amplified RF pulse to an electrosurgical instrument coupled to the output of the power converter.

13. The electrosurgical generator according to claim 9, wherein the pulsed electric field is an oscillating pulsed RF electric field including a DC offset.

14. The electrosurgical generator according to claim 9, wherein the power converter is configured to adjust a voltage amplitude duration of the pulsed electric field to control a degree of electroporation of the tissue.

15. The electrosurgical generator according to claim 9, wherein the power converter is configured to adjust a shape of the pulsed electric field to control a degree of electroporation of the tissue.

16. The electrosurgical generator according to claim 9, wherein the power converter is configured to adjust a number of pulses of the pulsed electric field to control a degree of electroporation of the tissue.

17. A method for sealing tissue, the method comprising:
positioning at least one electrode of an electrosurgical instrument in proximity to tissue;
applying a pulsed electric field to the at least one electrode of the electrosurgical instrument to electroporate the tissue;
grasping the electroporated tissue between a pair of jaw members of the electrosurgical instrument; and
delivering RF current to the grasped electroporated tissue via the at least one electrode of the electrosurgical instrument to seal the electroporated tissue.

18. The method according to claim 17, further comprising adjusting a voltage amplitude duration of the applied pulsed electric field to control a degree of electroporation of the tissue.

19. The method according to claim 17, further comprising adjusting a pulse shape of the applied pulsed electric field to control a degree of electroporation of the tissue.

20. The method according to claim 17, further comprising adjusting a number of pulses of the applied pulsed electric field to control a degree of electroporation of the tissue.
